(19) United States
(12) Patent Application Publication
(10) Pub. No.: US 2021/0391053 A1
(43) Pub. Date: Dec. 16, 2021

(54) PROVIDING NUTRITIONAL NEEDS FOR TREATMENT OF DISEASE STATES

(71) Applicant: Scriptrition LLC., Arvada, CA (US)

(72) Inventors: Matthew DePalo, Arvada, CO (US); Emily Williams, Wheat Ridge, CO (US)

(21) Appl. No.: 17/346,848

(22) Filed: Jun. 14, 2021

Related U.S. Application Data
(60) Provisional application No. 63/038,583, filed on Jun. 12, 2020.

(51) Int. Cl. G16H 20/60; G16H 50/30

(52) U.S. Cl. CPC G16H 20/60; G16H 50/30

(57) ABSTRACT

A method includes receiving, at a computing system, at least one health metric associated with a patient; receiving, at the computing system, information from a patient computing device corresponding to a meal box fulfillment method; categorizing the patient into a nutritional needs category based, at least in part, on the at least one health metric, the nutritional needs category being defined by nutritional criteria; determining a list of meals meeting the nutritional criteria of the nutritional needs category; providing the list of meals to the patient computing device; receiving, at the computing system, information from the patient computing device corresponding to selected meals from the list of meals; and based, at least in part, on receiving information corresponding to the selected meals, providing the information corresponding to the selected meals to a meal box fulfillment center computing device.

### CROSS-REFERENCE TO RELATED APPLICATIONS

The present application claims priority from U.S. Provisional Patent Application No. 53/038,583, METHODS OF PROVIDING NUTRITIONAL NEEDS FOR TREATMENT OF CARDIOVASCULAR DISEASE, filed on Jun. 12, 2020, the disclosure of which is hereby incorporated by reference in its entirety.

### BACKGROUND

Medications and medical procedures have long been used to treat health conditions and disease states, such as cardiovascular disease (CVD). People may look for alternatives to medications and procedures to treat and manage health conditions and disease states.

### SUMMARY

In one aspect, the technology relates to a method, comprising: receiving, at a computing system, at least one health metric associated with a patient; receiving, at the computing system, information from a patient computing device corresponding to a meal box fulfillment method; categorizing the patient into a nutritional needs category based, at least in part, on the at least one health metric, the nutritional needs category being defined by nutritional criteria; determining a list of meals meeting the nutritional criteria of the nutritional needs category; providing the list of meals to the patient computing device; receiving, at the computing system, information from the patient computing device corresponding to selected meals from the list of meals; based, at least in part, on receiving information corresponding to the selected meals, providing the information corresponding to the selected meals to a meal box fulfillment center computing device; and receiving, at the computing system, a meal box fulfillment confirmation message from the meal box fulfillment center computing device, the confirmation message including the information corresponding to the selected meals and an estimated shipment time of the meal box containing the selected meals. In an example, the nutritional needs category is a first nutritional needs category, and the method further comprises: receiving, at the computing system, at least one updated health metric after the patient has consumed at least one selected meal in the meal box; and categorizing the patient into a second nutritional needs category based, at least in part, on the at least one updated health metric. In an example, nutritional criteria associated with the second nutritional needs category is different than the nutritional criteria associated first nutritional needs category.

In an example, the at least one health metric is based, at least in part, on received prescription data associated with the patient. In another example, the at least one health metric comprises at least one disease state, age, gender, race, ethnicity, height, and weight. In yet another example, the disease state is one or more of cardiovascular disease, inclusive of peripheral arterial disease and electrophysiological diseases, Coronary disease, atrial fibrillation, heart failure, dyslipidemia, cardio metabolic disease, metabolic disease, type I diabetes, type II diabetes, obesity, hypertension, rheumatoid arthritis, osteoarthritis, orthopedic diseases, endocrine diseases, renal diseases, cancer, gut dysfunction, immune diseases, auto-immune diseases, genetic diseases, inflammatory diseases, gastrointestinal diseases, neurological diseases, urological diseases, cancers, pulmonary diseases, and dermatological diseases.

In an example, the list of meals is based, at least in part, on a combination of a determined geographic location of the patient and the information corresponding to the meal box fulfillment method. In another example, the information corresponding to the meal box fulfillment method comprises a selection of at least one of delivery to a delivery address or pick-up from an authorized pick-up location. In yet another example, the method further comprises receiving, at the computing system, a consumption message indicating the patient has consumed at least one meal from the meal box, the consumption message comprising a date and a time of consumption for the at least one meal. In still another example, the method further comprises providing a report to an insurance coverage provider, wherein the report comprises a cost associated with the meal box.

In another aspect, the technology relates to a system, comprising: a processor; and a memory communicatively coupled to the processor and storing instructions that, when executed by the processor, perform operations, comprising: receiving, at a computing system, at least one health metric associated with a patient; receiving, at the computing system, information from a patient computing device corresponding to a meal box fulfillment method; categorizing the patient into a nutritional needs category based, at least in part, on the at least one health metric, the nutritional needs category being defined by nutritional criteria; determining a list of meals meeting the nutritional criteria of the first nutritional needs category; providing the list of meals to the patient computing device; receiving, at the computing system, information from the patient computing device corresponding to selected meals from the list of meals; based, at least in part, on receiving information corresponding to the selected meals, providing the information corresponding to the selected meals to a meal box fulfillment center computing device; and receiving, at the computing system, a meal box fulfillment confirmation message from the meal box fulfillment center computing device, the confirmation message including the information corresponding to the selected meals and an estimated shipment time of the meal box containing the selected meals.

In an example, the memory communicatively coupled to the processor and storing instructions that, when executed by the processor, perform operations, further comprising: receiving, at the computing system, at least one updated health metric after the patient has consumed at least one selected meal in the meal box; and categorizing the patient into a second nutritional needs category based, at least in part, on the at least updated health metric. In another example, nutritional criteria associated with the second nutritional needs category is different than the nutritional criteria associated first nutritional needs category. In yet another example, the at least one health metric is based, at least in part, on received prescription data associated with the patient.

In an example, the at least one health metric comprises at least one disease state, age, gender, race, ethnicity, height, and weight. In another example, the disease state is one or more of cardiovascular disease, inclusive of peripheral arterial disease and electrophysiological diseases, Coronary disease, atrial fibrillation, heart failure, dyslipidemia, cardio metabolic disease, metabolic disease, type I diabetes, type II diabetes, obesity, hypertension, rheumatoid arthritis, osteoarthritis, orthopedic diseases, endocrine diseases, renal diseases, cancer, gut dysfunction, immune diseases, auto-immune diseases, genetic diseases, inflammatory diseases, gastrointestinal diseases, neurological diseases, urological diseases, cancers, pulmonary diseases, and dermatological diseases.

In an example, the list of meals is based, at least in part, on a combination of a determined geographic location of the patient and the information corresponding to the meal box fulfillment method. In another example, information corresponding to the meal box fulfillment method comprises a selection of at least one of delivery to a delivery address or pick-up from an authorized pick-up location. In yet another example, the memory communicatively coupled to the processor and storing instructions that, when executed by the processor, perform operations, further comprising: receiving, at the computing system, a consumption message indicating the patient has consumed at least one meal from the meal box, the consumption message comprising a date and a time of consumption for the at least one meal.

In another aspect, the technology relates to a method, comprising: receiving, at a computing system, at least one health metric associated with a patient; receiving, at the computing system, information from a patient computing device corresponding to a meal box fulfillment method; categorizing the patient into a first nutritional needs category based, at least in part, on the at least one health metric, the nutritional needs category being defined by nutritional criteria; determining a list of meals meeting the nutritional criteria of the first nutritional needs category; based, at least in part on the selected meal box fulfillment method, altering the list of meals; providing the list of meals to a patient computing device; based, at least in part, on receiving, at the computing system, information corresponding to selected meals from the list of meals, updating the list of meals provided to the patient computing device; receiving, at the computing system, at least one updated health metric after the patient has consumed at least one selected meal in the meal box; and categorizing the patient into a second nutritional needs category based, at least in part, on the at least one updated health metric.

### BRIEF DESCRIPTION OF THE DRAWINGS

Non-limiting and non-exhaustive examples are described with reference to the following Figures.

FIG. 1A illustrates a system for analyzing a health metric associated with a patient to determine a meal program according to an example.

FIG. 1B illustrates the system of FIG. 1A in which a list of meals are provided according to an example.

FIG. 1C illustrates the system of FIG. 1A in which selected meals are provided according to an example.

FIG. 1D illustrates the system of FIG. 1A in which a confirmation message is provided according to an example.

FIG. 2 illustrates an example meal including nutritional information according to an example.

FIG. 3 illustrates a comparative nutrient listing of the example meal in FIG. 2 according to an example.

FIG. 4 illustrates a method for analyzing a health metric associated with a patient to determine a meal program according to an example.

FIG. 5 is a system diagram of various components of a computing device according to an example.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Cardiovascular disease (CVD) effects many Americans. As discussed briefly above, treatment of certain disease states, such as CVD, typically involves use of medications. However, dietary changes may also reduce rates of CVD. But dietary changes can be difficult to make. If food intake can be managed for patients through the use of personalized meal programs, where meal provisions are provided directly to patients, dietary changes may be made more easily. For example, meal programs may be customized for the particular needs of an individual patient based on his or her health information to assist in the treatment of the patient. A patient's health condition or disease state may improve if the patient consumes meals having certain nutritional criteria, where the nutritional criteria is selected based on the patient's health information and health metrics.

The methods and systems described herein provide a patient with reasonable access to healthy foods. A patient may be an individual, including an individual with a medical condition or disease state. In examples, a patient may have more than one medical condition or disease state. Providing healthy foods, which contain nutritional criteria calculated specifically for a patient, to the patient may include home-delivered or pick-up-ready personalized meal boxes developed by medical professionals and individualized to each patient based on the patient's health metrics. Each personalized meal box may include a plurality of meals for a patient for a predetermined treatment period. Every meal within a meal box may have the foundation of being structured from input of leading field researchers and a compilation of information from many sources, including but not limited to the American College of Cardiology, the American Heart Association, American Cancer Society, and American Diabetes Association nutritional recommendations and guidelines. The meals may be provided in proportions approved by medical providers, and prepared and/or formulated by professional chefs.

A meal box may be customized for the particular needs of an individual patient based on his or her health metrics to assist in treatment of a diseases state, such as CVD. The contents of a meal box may be determined based on receiving a variety of inputs associated with a patient's health metrics. The health metrics are used to determine which nutrients, and in which amounts, are needed for the patient. A meal box, which includes a plurality of prepared meals, is generated according to the specific nutritional needs of the patient. For example, a meal box may be prepared with varying amounts of ingredients to provide the targeted amounts of various nutrients for a particular patient. A patient's health condition or disease state may improve if the patient consumes the meals from the meal box having certain nutritional criteria for a treatment period. As a patient's disease state improves and health metrics change, the patient's nutritional needs may also change. The nutrients in a personalized meal box can change as the patient's nutritional needs change.

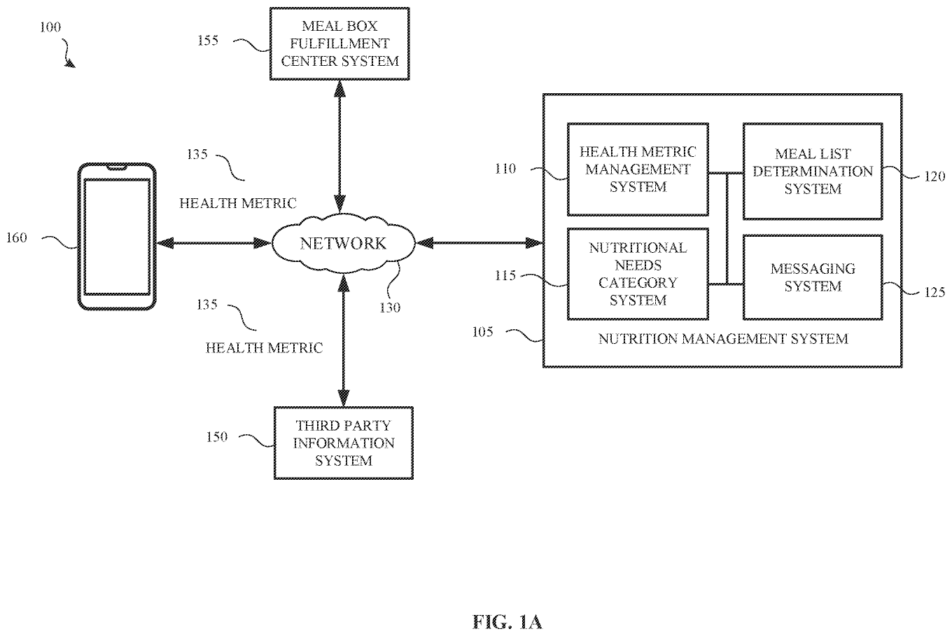

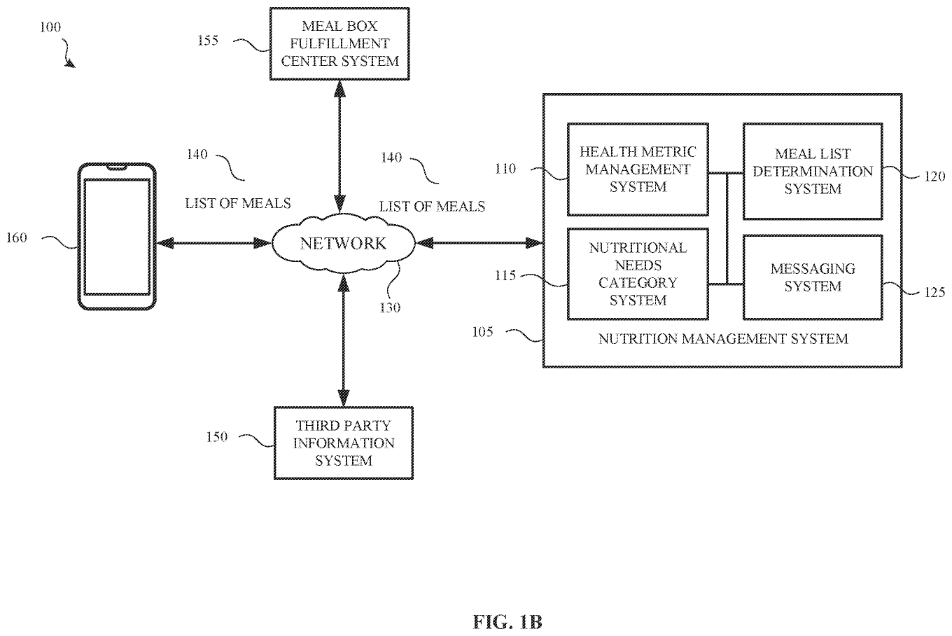

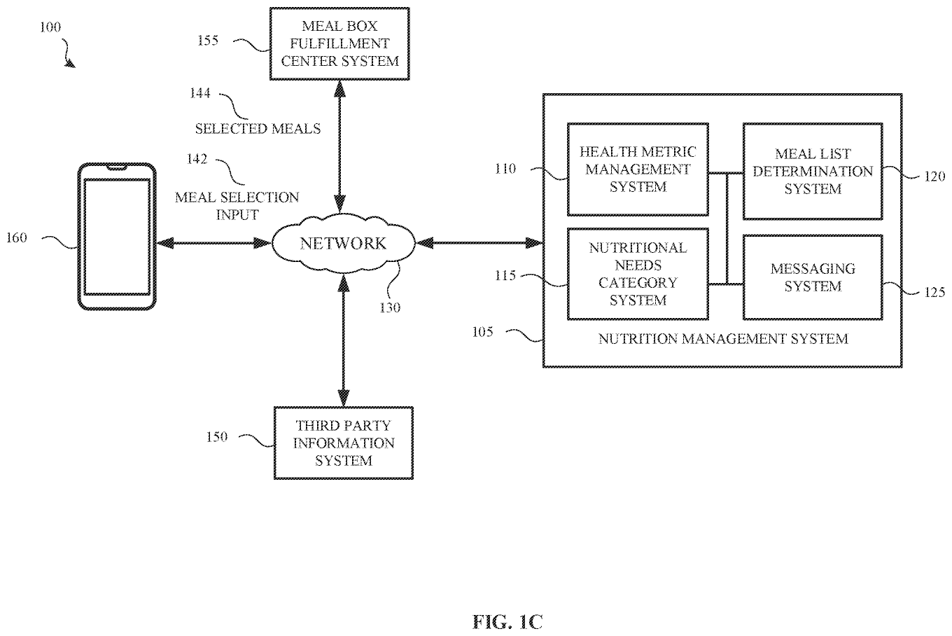

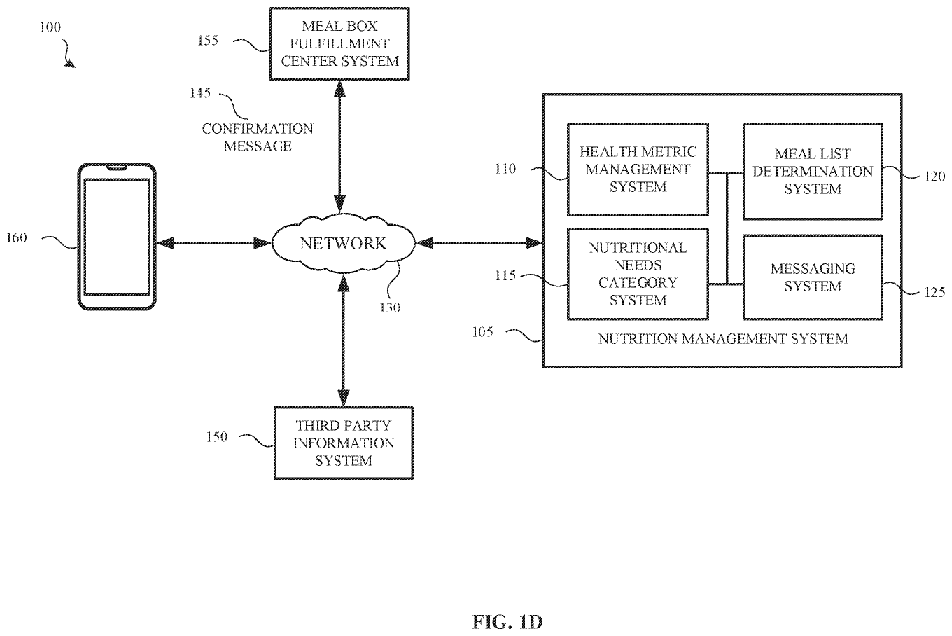

These and other features will be further described below with respect to FIG. 1A-FIG. 5.

FIG. 1A illustrates a system 100 for analyzing a health metric 135 associated with a patient to determine a meal program containing meals that meets nutritional needs of the patient according to an example. To determine a meal program that meets certain nutritional needs, the system 100 includes a nutrition management system 105. The nutrition management system 105 includes a health metric management system 110, a nutritional needs category information system 115, a meal list determination system 120, and a messaging system 125. The system 100 further includes a network 130, which may be in communication with a third party information system 150, and a meal box fulfillment center system 155.

In examples, the nutrition management system 105 may receive at least one health metric 135 associated with a patient. The health metric 135 may be received, via a network 130, from a computing device 160 associated with the patient. For example, a patient may manually input a health metric 135 on the patient computing device 160. An application executing on the patient computing device 160 may then transmit the health metric 135 to the nutrition management system 105 over the network 130. In other examples, a health metric may be received via sensor information, such as, but not limited to, heart rate sensor information, blood glucose sensor information, or other health information that may be read or determined by a sensor.

In another example, the health metric 135 may be received, via the network 130, from a third party information system 150. The third party information system 150 may be a prescription data information system or other type of information system that identifies, detects, and/or otherwise receives and stores prescription data associated with a patient. In examples, prescription data associated with a patient can be sent by a clinician or physician to the third party information system 150. In other examples, the third party information system 150 may be a laboratory data information system or other type of information system that identifies, detects, and/or otherwise receives and stores laboratory data associated with a patient. In even other examples, the third party information system 150 may be an electronic medical records system or other type of information system that identifies, detects, and/or otherwise receives and stores medical records associated with a patient.

For example, one or more laboratory tests (e.g., a blood test, a cardio metabolic panel, an inflammatory panel) and/or procedures may be performed on the patient, with the patient's consent, to monitor the status of a particular disease state. Results and information from such laboratory tests may be transmitted from the third party information system 150, over the network 130, to the nutrition management system 105. Once the at least one health metric 135 is received by the nutrition management system 105, the health metric management system 110 stores the at least one health metric 135. The at least one health metric 135 may be encrypted and securely stored. In another example, the health metric 135 may be temporarily stored and subsequently deleted so as to retain patient confidentiality. In yet another example, a patient may opt out of providing any health information.

A health metric 135 may be any information associated with a patient. In examples, a health metric 135 can include, but is not limited to, personal information (e.g., name, birthdate, address), and medical information associated with a patient. Medical information associated with a patient may include, but is not limited to, a disease state, age, gender, race, ethnicity, height, weight, and allergy information associated with the patient. Example disease states include, but are not limited to cardiovascular disease, inclusive of peripheral arterial disease and electrophysiological diseases, Coronary disease, atrial fibrillation, heart failure, dyslipidemia, cardio metabolic disease, metabolic disease, type I diabetes, type II diabetes, obesity, hypertension, rheumatoid arthritis, osteoarthritis, orthopedic diseases, endocrine diseases, renal diseases, cancer, gut dysfunction, immune diseases, auto-immune diseases, genetic diseases, inflammatory diseases, gastrointestinal diseases, neurological diseases, urological diseases, cancers, pulmonary diseases, and dermatological diseases. In examples, a health metric may include laboratory data associated with a patient. In other examples, a health metric may include risk factors associated with a patient. In still other examples, a health metric may include an activity level associated with the patient. An activity level may be indicated generally (e.g., low, medium, high) or more specifically, with type and frequency of activity (e.g., 3 mile run, 4 times per week). A health metric 135 may further include a health goal associated with the patient. Examples of health goals may include, but are not limited to disease reversal, disease improvement, risk factor reduction, or weight loss.

In some examples, an updated health metric may be provided to the nutrition management system 105 at any point after an initial health metric 135 is received. In examples, the updated health metric may be provided to the health metric management system 110 after the patient has consumed at least one meal in the meal box. The updated health metric may be the same or different from the first at least one health metric received by the health metric management system 110. As with the at least one health metric, the updated health metric may be received, over the network 130, from a patient computing device 160 or from a third party information system 150. The patient computing device 160 or third party information system 150 may provide an updated health metric to the nutrition management system 105 voluntarily. Alternatively, the nutrition management system 105 may provide a request for an updated health metric to either the patient computing device 160 or the third party information system 150. In examples, the nutrition management system 105 may continuously access a third party information system 150 and, as the third party information system 150 updates the at least one health metric associated with the patient, the health metric management system 110 will automatically receive and store the updated at least one health metric such that the most recent updated health metrics associated with a patient are stored in the health metric management system 110.

An example updated health metric may be an updated weight associated with the patient. For example, the weight of the patient may have been one hundred and forty pounds, and the updated weight of the patient may be one hundred and thirty eight pounds. The updated health metric may be stored in the health metric management system 110. In examples, the updated health metric may be compared to an anticipated or predicted updated health metric generated by the health metric management system 110. The predicted updated health metric may be based on expected results from a patient's consumption of meals in a meal box for a predetermined amount of time. If the health metric management system 110 determines the updated health metric differs from the predicted updated health metric, the nutrition management system 105 may adjust the target nutritional criteria for the patient.

FIG. 1B illustrates the system of FIG. 1A in which a list of meals 140 are provided to a patient computing device according to an example. Once a health metric is received by the nutrition management system 105 and stored in the health metric management system 110, the nutritional needs category system 115 may categorize a patient into a nutritional needs category. The nutritional needs category information system 115 stores information associated with predetermined nutritional needs categories, where each nutritional needs category is defined by separate and distinct target nutritional criteria.

A patient may be categorized into one of the nutritional needs category based on the received at least one health metric associated with the patient. In some cases, where a plurality of health metrics associated with a patient are received by the nutrition management system 105, the patient may be categorized into a nutritional needs category based on a combination of the plurality of received health metrics. In examples, the nutritional needs category system 115 may access health metrics stored by the health metric management system 110. Using the health metrics stored in the health metric management system 110, the nutritional needs category system 115 may determine which category the patient should be categorized into. The nutritional needs category system 115 may also access updated health metrics and predicted updated health metrics stored by the health metric management system 110. The updated health metrics and predicted updated health metrics may be used by the nutritional needs category system 115 to categorize a patient into a nutritional needs category based, at least in part, on how closely the patient's updated health metrics match the predicted updated health metrics.

For example, each separate category may have a body mass index (BMI) range. Based on a patient's height and weight stored in the health metric management system 110, the patient's BMI may be calculated. The patient may then be categorized into a category based, at least in part, on his or her BMI. In other examples, there may be separate categories for males and females. The nutritional needs category system 115 may categorize the patient into a category based, at least in part, on gender. For example, there may be a set of categories for male patients (e.g., male specific categories A-Z), and a separate set of categories for female patients (e.g., female specific categories A-Z). In non-limiting examples, there may be 50 categories, 100, categories, or 200 categories. Other health metrics, including but not limited to a disease state or a laboratory test result, may also be used to categorize the patient into a nutritional needs category.

In examples, there may be subcategories within each nutritional needs category. A patient may be categorized into a subcategory of a nutritional needs category based, at least in part, upon the nutrition management system 105 receiving a category modifier. A category modified may be, but is not limited to, an allergy (e.g., peanut allergy), a dietary restriction (e.g., gluten intolerant), or a dietary preference (e.g., prefers no mushrooms). For example, the nutrition management system 105 may receive a category modifier from the patient computing device 160 indicating that the patient has a peanut allergy. The patient may then be subcategorized into a sub-nutritional needs category defined by nutritional criteria having no nuts.

Each separate nutritional needs category is defined by target nutritional criteria. For example, each separate category may have a daily calorie range, the daily calorie range being the targeted number of calories a patient categorized into that nutritional needs category should consume per day. Additionally, each separate category may have further target nutritional criteria for a patient categorized into that nutritional needs category. Such additional nutritional criteria includes target quantities of, but not limited to, carbohydrates, vegetables, fruits, protein, dairy, fiber, sugar, total fat, saturated fat, and sodium. In examples, the target nutritional criteria for a nutritional needs category may be a range. For example, a target daily calorie intake may be 2,000 calories, plus or minus 200 calories (i.e., 1,800-2,200 calories per day).

The nutritional needs category the patient is categorized into may be a first nutritional needs category defined by first nutritional criteria. In some cases, based, at least in part, on receiving an updated health metric, the nutritional needs category system 115 may categorize the patient into a second nutritional needs category defined by second nutritional criteria. The first nutritional needs category may be different or the same as the second nutritional needs category. Where the first nutritional needs category is different from the second nutritional needs category, the first nutritional criteria is different from the second nutritional criteria. In examples, if an updated health metric differs from a predicted updated health metric, the nutritional needs category system 115 may determine that the patient is not responding as predicted to a meal box having certain nutritional criteria and therefore the patient may be subsequently categorized into a different nutritional needs category being defined by different nutritional criteria.

The nutrition management system 105 may also include a meal list determination system 120. The meal list determination system 120 accesses and stores meals and the nutritional information associated with each meal. Such nutritional information may include the ingredients within each meal and a breakdown of the nutrition facts for each meal (e.g., calories, total fat, protein, sodium, sugar). In examples, the meals stored in the meal list determination system 120 have highly adjustable ingredient lists. Therefore, the same basic recipe may be utilized for a variety of patients in different nutritional needs categories, while altering individual ingredient amounts to provide the patient with the necessary nutritional criteria. The meal list determination system 120 may access external databases containing a listing of meals to continually update the meals and nutritional information stored in the meal list determination system 120.

The nutrition management system 105 may determine, using the meal list determination system 120, a list of meals meeting the target nutritional criteria of the nutritional needs category that the patient has been categorized into. After a health metric has been received and the patient has been categorized into a nutritional needs category, the meal list determination system 120 may parse through the meals and nutritional information and determine which meals meet the nutritional criteria associated with the nutritional needs category. The meal list determination system 120 may then determine a list of meals meeting the patient's specific nutritional needs. A list of meals may meet the nutritional criteria of the nutritional needs category if the nutritional information of the meals on the list of meals combined falls within the range of targeted nutritional criteria for the nutritional needs category. The list of meals 140 may be provided from the meal list determination system 120, through the network 130, to the patient computing device 160.

In examples, the nutrition management system 105 may receive, from the patient computing device, information corresponding to a meal box fulfillment method. The meal box fulfillment method may be, but is not limited to, delivery to a delivery address or pick-up from an authorized pick-up location. For example, the patient may choose to have his or her meal boxes delivered to a home address, or to a work address. In other examples, the patient may choose to pick-up his or her meal boxes at an authorized pharmacy or other authorized distribution center (e.g., grocery store, wholesaler, health clinic). Based on the received information corresponding to a meal box fulfillment method, the meal list determination system 120 may alter the list of meals provided to the patient computing device 160. For example, certain meals may only be available if a patient selects a delivery fulfillment method, and other meals may only be available if the patient selects a pick-up fulfillment method.

In examples, the number of meals on the list of meals 140 provided from the nutrition management system 105 may be a specified number of meals per day for a predetermined treatment period. The specified number of meals per day may range from 1 meal per day to 5 meals per day. The specified number of meals per day may also include snacks to aid in meeting the target nutritional criteria of a patient in a given day. For example a patient may be prescribed 4 meals and 2 snacks per day. The predetermined treatment period may range from 1-1100 days. The predetermined treatment period may be renewed, reset, altered, or continued in order to modify or adjust the predetermined treatment period at any point during a treatment period or upon the conclusion of a treatment period. In other examples, the list of meals 140 may include a number of meals greater than a specified number of meals per day for a predetermined treatment period. For example, if a patient has been prescribed twenty meals to consume in a five day treatment period, the patient may be provided a list of thirty meals, with instructions to select twenty meals for his or her meal box. This enables a patient to select a subset of the meals within the list of meals to be included in his or her meal box. In this example, by receiving a selection of meals from the patient, there is a higher likelihood that the patient will enjoy the meals in the meal box. The selection of a subset of meals within the provided list of meals will be described in more detail below, for example, in FIG. 1C.

The meal list determination system 120 may be in communication with a supply chain information system. The supply chain information system may receive and store supply chain information for geographic areas. In examples, the nutrition management system 105 may receive a health metric associated with a patient that includes a geographic location of the patient. The supply chain information system may access supply chain information for the specific geographic location of the patient, and the list of meals determined by the meal list determinations system 120 may be based, in part, on such supply chain information. For example, two patients categorized into the same nutritional needs category, where one patient is located in Florida and the other patient is located in Oregon, may receive a different list of meals due, in part, to their different geographic locations within the supply chain. Basing the list of meals, in part, on a patient's geographic location and the supply chain information of that geographic location may lower the cost of providing a meal box to a patient and ensure fresh ingredients are contained within each meal box. In examples, the list of meals provided to the patient computing device may be based, in part, on a combination of the patient's geographic location and the received information corresponding to the meal box fulfillment method.

FIG. 1C illustrates the system of FIG. 1A in which selected meals are provided and sent to a fulfillment center according to an example. For example and referring to FIG. 1C, after a list of meals has been provided to a patient computing device 160, the patient may view the list of meals. The patient may then select meals from the list of meals he or she would like included in a meal box. The meal selection input 142 may then be provided, over the network 130, to the nutrition management system 105. The messaging system 125 may receive and store the selected meals. In examples where the list of meals provided from the nutrition management system 105 contains a number of meals greater than a specified number of meals for a predetermined treatment period, the meal selection input 142 includes information corresponding to only a subset of meals from the list of meals. For example, if a meal box is to include three meals per day for a predetermined treatment period of five days (i.e., the meal box will include fifteen meals), the nutrition management system 105 may provide a list of twenty meals to the patient computing device 160 with instructions to select fifteen meals. When viewing the meals in the list of meals provided, the patient may view the nutritional information and nutrition facts of each meal to assist with his or her decision regarding which meals to select for inclusion in his or her meal box.

The meal list determination system 120 may arrange meals into a personalized meal program through cumulative totaling of the nutritional contents of all the meals within a meal box. As a patient selects meals from the list of meals he or she wants included in a meal box, the meal selection input 145 is provided from the patient computing device 160, over the network 130, to the nutrition management system 105 in real time on a meal-by-meal basis. Thus, as soon as a patient selects one meal, information corresponding to that meal selection may be provided to the nutrition management system 105. The meal list determination system 120 may then update the cumulative total of the nutritional contents in the meal box and eliminate meals from the provided list of meals that no longer fit the patient's target nutritional criteria. For example, based on a patient selecting one meal from the list of meals, the meal list determination system 120 may determine that certain other meals originally provided on the list of meals, in combination with the one selected meal, will no longer meet the target nutritional needs of the patient.

In these examples, based, at least in part, on the received meal selection input 145, the meal list determination system 120 may provide an updated list of meals to the patient computing device 160 for the patient to select meals from. As the meal list determination system 120 receives additional meal selection input 145, the meal list determination system 120 may be concurrently calculating a cumulative total of nutritional contents based on the nutritional contents of each meal selected by the patient. For example, a patient may select a meal from the provided list of meals that has a high amount of sodium. Upon receiving meal selection input 145 corresponding to the high-sodium meal, the meal list determination system 120 may eliminate other high-sodium meals from the provided list of meals to ensure that the total amount of sodium within the meal box (i.e., within all the meals in the meal box combined) falls in the target range of sodium targeted for the patient. The meal list determination system 120 may arrange meals into a personalized meal box in this manner with or without altering the nutritional contents of the meals to provide a meal box containing a patient's target nutritional criteria.

In examples, once the messaging system 125 has received and stored the meal selection input 142 from the patient computing device 160, the messaging system 125 may provide information corresponding to the selected meals 144, over the network 130, to a meal box fulfillment center system 155. The meal box fulfillment center system 155 may be associated with a meal box fulfillment center, such as a food distribution center having the ability to prepare and ship a meal box to a patient. In other examples, the meal box fulfillment center system 155 may use a third party delivery service to ship and deliver meal boxes.

FIG. 1D illustrates the system of FIG. 1A in which a confirmation message is transmitted from a meal box fulfillment center system according to an example. The meal box fulfillment center system 155, after receiving the information corresponding to the selected meals, may provide a meal box fulfillment confirmation message 145 to confirm it will prepare the selected meals for a meal box. The meal box fulfillment confirmation message 145 may be sent from the meal box fulfillment center 155, over the network 130, to the nutrition management system 105. The messaging system 125 may receive and store the meal box fulfillment confirmation message 145. The meal box fulfillment confirmation message 145 may include information corresponding to the selected meals and an estimated ship time of a meal box. Specifically, the meal box fulfillment confirmation message may include information to identify each of the selected meals, and it may further include an estimated ship date when the meal box will be prepared and ready for shipment to the patient.

The meal boxes may be associated with a delivery service. The meals can be packaged in a way that preserves the freshness of the meal, allows for easy meal assembly, and arrives at the patient's residence or place of preference. Delivery can be delivered every day, every other day, every 3 days, every 4 days, weekly, bi-weekly, or monthly. Delivery of meal boxes saves the patient time and effort. In other examples, a meal box may be picked-up at a store or pharmacy. In even other examples, a meal box may be provided to patients in hospitals, or provided in hospital cafeterias for patients, patient guests, or employees.

In some examples, the patient may keep track of his or her meal consumption to assist with the treatment of a health condition or disease state. As part of the patient's meal tracking, the nutrition management system 105 may receive a consumption notification from the patient computing device 160 indicating the patient has consumed at least one meal from the meal box. The consumption notification may identify what meal was consumed, and include a date and time of consumption for the at least one meal. In examples, each meal within the meal box may contain a barcode or other identification code on the packaging. The consumption notification may include an image of the barcode or identification code. For example, the patient may, upon consuming a meal, capture an image of the barcode or identification code using the patient computing device 160 and provide the image to the nutrition management system 105. The messaging system 125 may then receive and process the consumption notification containing the captured image, and store information related to the meal consumption. If, in examples, the messaging system 125 receives no consumption notifications from the patient computing device 160, the messaging system 125 may provide a consumption request notification to the patient computing device 160 requesting information corresponding to meals consumed by the patient.

Throughout a predetermined treatment period of a patient, the nutrition management system 105 may receive, collect, and store information associated with the patient, including but not limited to initial health metrics associated with the patient, updated health metrics associated with the patient, and a complete list of meals provided to the patient in his or her personalized meal boxes. In examples, with a patient's consent, the messaging system 125 may provide such information to an external database for storage and further analysis. For example, the external database may compile and analyze information from a plurality of patients to identify trends and correlations between health metrics, meals, and nutritional information. In examples, such trends and correlations from a plurality of patients may be used to optimize treatment of patients.

In examples, the nutrition management system 105 may provide a report to an insurance coverage provider. The report may be generated by the messaging system 125. The report may comprise a cost associated with the meal box. The messaging system 125 may provide the report, through the network 130, to a third party information system 150, where the third party information system 150 is an insurance coverage provider system. In examples, the insurance coverage provider may cover the total cost or a portion of the cost of a personalized meal box for a patient.

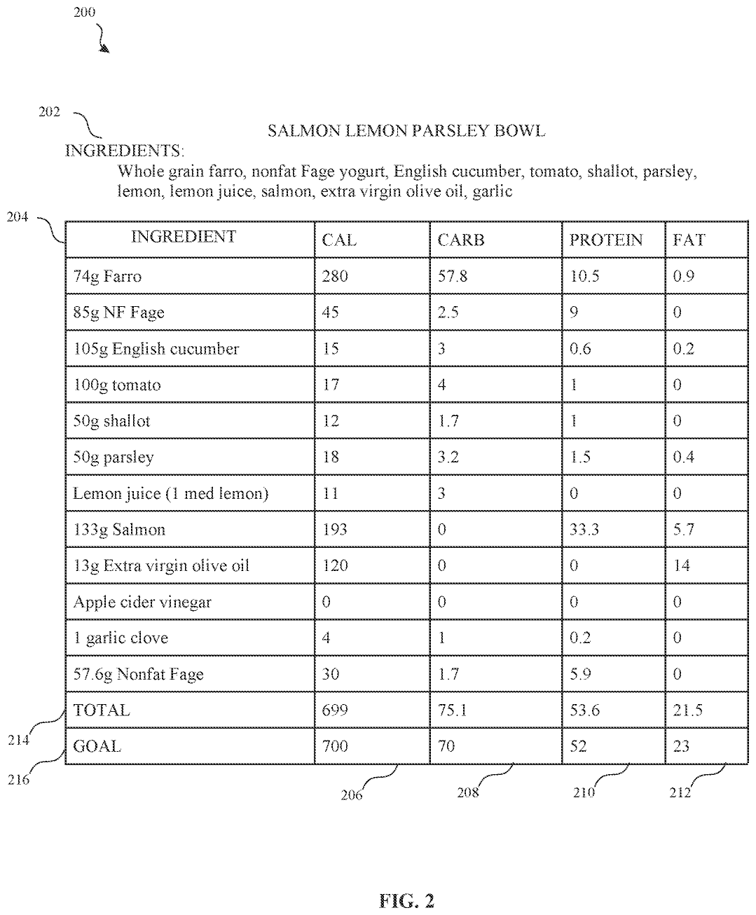

FIG. 2 illustrates an example meal 200 including nutritional information 204 according to an example. The example meal 200 may be one of the selected meals in the meal box described above in reference to FIGS. 1A-1D. As shown, the meal 200 is a salmon lemon parsley bowl. The meal 200 may include an ingredient list 202, which lists every ingredient in the meal. Nutritional information 204 may also be provided for each ingredient in the meal 200. The nutritional information 204 includes a caloric content 206, carbohydrate content 208, protein content 210, and fat content 212. Further, totals 214 of each of these groups are provided, as well as a goal amount 216. A patient may view the example meal 200 and nutritional information 204 for each meal on a list of meals to assist with selecting meals to be included in a meal box. When a patient receives a meal box, nutritional information 204 may be provided for each meal in the meal box in the format shown in FIG. 2.

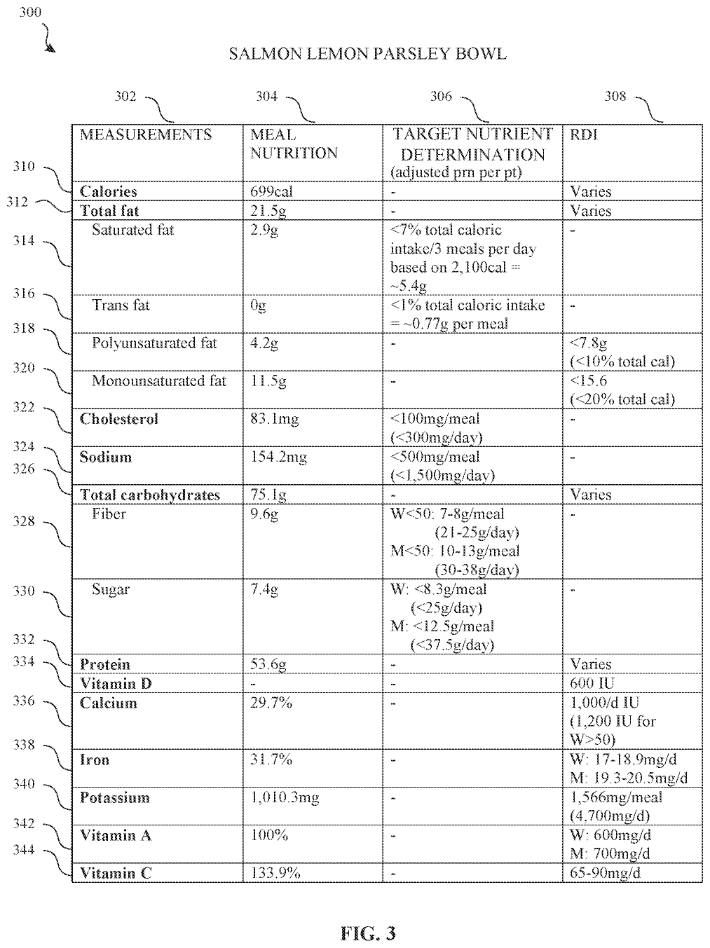

FIG. 3 illustrates a comparative nutrient listing 300 of the example meal in FIG. 2 according to an example. The nutrient listing 300 includes detailed information about the content of the meal. For example, measurements 302 may be provided, including but not limited to calories 310, total fat 312, cholesterol 322, sodium 324, total carbohydrates 326, protein 322, Vitamin D 334, Calcium 336, Iron 338, Potassium 340, Vitamin A 342, and Vitamin C 344. The total fat 312 may include a breakdown of the type of fats, including saturated fat 314, trans-fats 316, polyunsaturated fats 318, and monounsaturated fats 320. The carbohydrate content 326 may also include a breakdown showing further details such as fiber 328 and sugar 330 content. The amount of each nutrient of the meal 304 may also be provided, as well as a target nutrient determination 306 tailored to the patient, and the RDI amount 308.

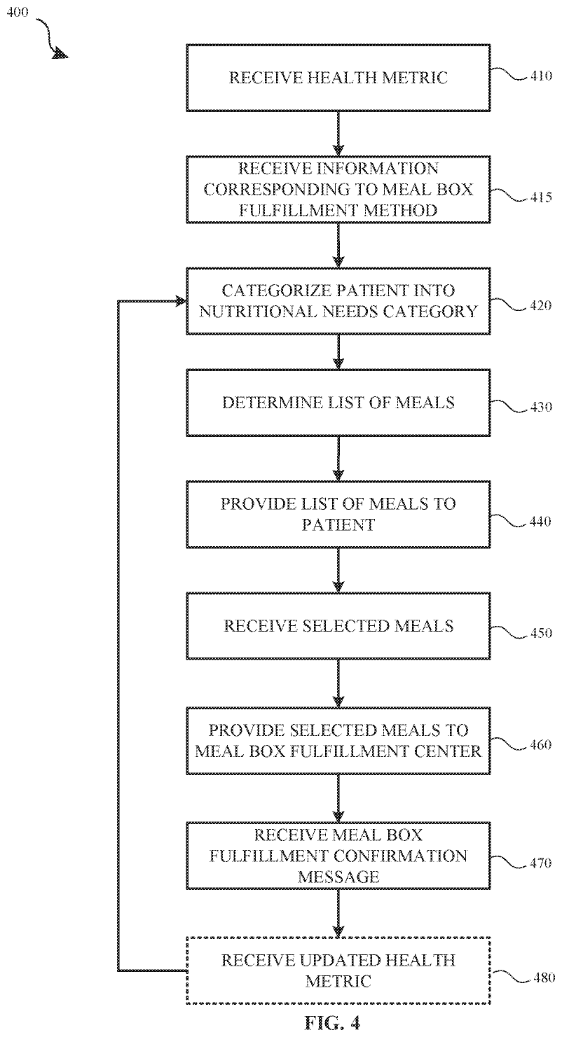

FIG. 4 illustrates a method 400 for analyzing a health metric associated with a patient to determine a meal program according to an example. The method 400 may be performed by the system 100 as described above, for example, in FIGS. 1A-1D. The method 400 includes, at operation 410, receiving, at a computing system, at least one health metric associated with a patient. An example system consistent with operation 410 is described above, for example, in FIG. 1A. The at least one health metric may be received from a patient computing device or a third party information system, such as a prescription data information system or laboratory data information system. The at least one health metric may be any information associated with a patient, including, but not limited to, personal information (e.g., name, birthdate, address), and medical information associated with the patient.

The method 400 may continue with receiving, at the computing system, information corresponding to a meal box fulfillment method from a patient computing device, operation 415. Information corresponding to a meal box fulfillment method may include, but is not limited to, a selection of at least one of delivery to a delivery address or pick-up from an authorized pick-up location.

Once the at least one health metric associated with the patient has been received at operation 410, the patient is categorized into a nutritional needs category at operation 420, based at least in part, on the received at least one health metric. The nutritional needs category is defined by nutritional criteria. The nutritional needs categories are described in more detail above, for example, in FIG. 1B. At operation 430, a list of meals is determined that meets the target nutritional criteria of the nutritional needs category the patient has been categorized into. In examples, meals and nutritional information associated with each meal are stored in a meal list determination system, as described above, for example, in FIG. 1B. The meal list determination system may then parse through the meals and corresponding nutritional information and determine a list of meals meeting the nutritional criteria associated with the nutritional needs category.

At operation 440, the list of meals is provided to the patient via the patient computing device. An example system consistent with operation 440 is described above, for example, in FIG. 1B. After the list of meals has been provided to the patient computing device, the patient may view the list of meals and nutritional information associated with each meal. The patient may then select meals from the list of meals he or she would like included in a personalized meal box. At operation 450, the computing system receives the information from the patient computing device corresponding to the selected meals from the list of meals. In certain examples, based at least in part on receiving information corresponding to selected meals on a meal-by-meal basis, the list of meals provided to the patient may be updated in real time, as described above in more detail, for example, with respect to FIG. 1C. Updating the list of meals provided to the patient based on receiving information corresponding to selected meals may ensure the meal box contains a total amount of nutrients across all the meals combined falling within the target range of nutritional criteria for the nutritional needs category the patient is in.

In response to receiving information corresponding to the selected meals, the information corresponding to the selected meals is provided to a meal box fulfillment center computing device, operation 460. An example system consistent with operations 450 and 460 is described above, for example, in FIG. 1C.

At operation 470, a meal box fulfillment confirmation message may be transmitted from the meal box fulfillment center system. An example system consistent with operation 470 is described above, for example, in FIG. 1D. After receiving the information corresponding to the selected meals, the meal box fulfillment center may provide a meal box fulfillment confirmation message to confirm it will prepare the selected meals for a meal box. The meal box fulfillment confirmation message may include information corresponding to the selected meals and an estimated ship time of the meal box.

In examples, at operation 480 an updated health metric may be provided. The updated health metric may be provided at any point after an initial health metric is received. The updated health metric may be provided after the patient has consumed at least one selected meal in the meal box. Based, at least in part on receiving the updated health metric, the patient may once again be categorized into a nutritional needs category, operation 420. In some examples, the patient is categorized into a first nutritional needs category based on the received health metric, and then the patient is categorized into a second nutritional needs category based on the received updated health metric. The first nutritional needs category may be different or the same as the second nutritional needs category. Where the first nutritional needs category is different from the second nutritional needs category, the first nutritional criteria is different from the second nutritional criteria. The process of receiving an updated health metric and categorizing the patient into a nutritional needs category based, at least in part on the updated health metric, may continue throughout a predetermined treatment period or until a physician determines a patient no longer requires treatment. For example, a patient may be further categorized, based at least in part on an updated health metric, into a third nutritional needs category, a fourth nutritional needs category, and a fifth nutritional needs category, wherein each nutritional needs category is defined by different nutritional criteria.

In some examples, when a patient is categorized into a second nutritional needs category different from the first nutritional needs category, the meals in the meal box meeting the second nutritional criteria may be different from the meals meeting the first nutritional criteria. Thus, as a patient continues to consume meals from personalized meal boxes over time, and the patient's health metrics change as a disease state improves, the patient can continue receiving adequate nutrients based on his or her most current health metrics.

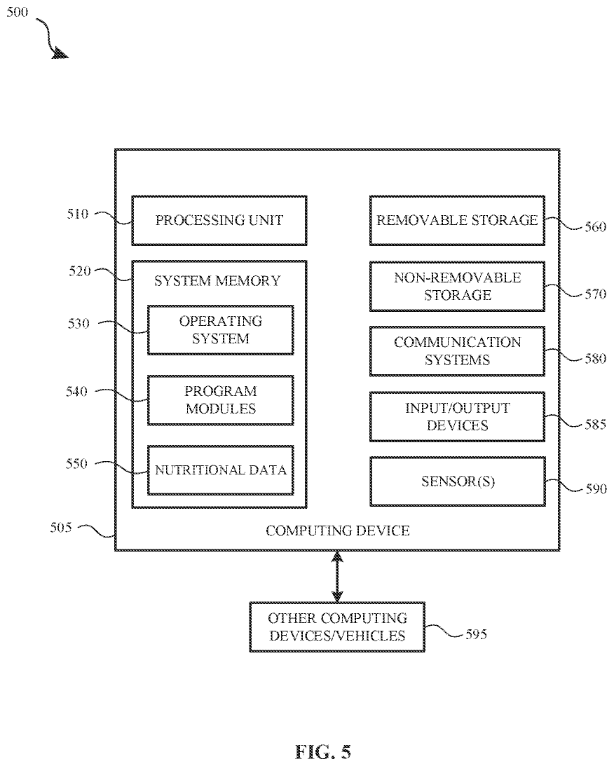

FIG. 5 is a system diagram of a computing device 500 according to an example. The computing device 500 may be associated or otherwise integrated with the various systems shown and described with respect to FIGS. 1A-1D. As shown in FIG. 5, the physical components (e.g., hardware) of the computing are illustrated and these physical components may be used to practice the various aspects of the present disclosure.

The computing device 500 may include at least one processing unit 510 and a system memory 520. The system memory 520 may include, but is not limited to, volatile storage (e.g., random access memory), non-volatile storage (e.g., read-only memory), flash memory, or any combination of such memories. The system memory 520 may also include an operating system 530 that control the operation of the computing device 500 and one or more program modules 540. The program modules 540 may be responsible for gathering environmental condition information, light electric vehicle feature and capability information, rider profile information and so on. The memory may also store this received/determined information 550 or otherwise provide access to this information.

The computing device 500 may also have additional features or functionality. For example, the computing device 500 may include additional data storage devices (e.g., removable and/or non-removable storage devices) such as, for example, magnetic disks, optical disks, or tape. These additional storage devices are labeled as a removable storage 560 and a non-removable storage 570.

A number of different program modules and data files may be stored in the system memory 520. While executing on the processing unit 510, the program modules 540 may perform the various processes described above.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 5 may be integrated onto a single integrated circuit. Such a SOC device may include one or more processing units, graphics units, communications units, system virtualization units and various application functionality all of which are integrated (or “burned”) onto the chip substrate as a single integrated circuit.

When operating via a SOC, the functionality, described herein, may be operated via application-specific logic integrated with other components of the electric vehicle 500 on the single integrated circuit (chip). The disclosure may also be practiced using other technologies capable of performing logical operations such as, for example, AND, OR, and NOT, including but not limited to mechanical, optical, fluidic, and quantum technologies. In addition, examples of the disclosure may be practiced using a computing device associated with or integrated with the electric vehicle and/or in any other circuits or systems.

The computing device 500 may include one or more communication systems 580 that enable the electric vehicle to communicate with rechargeable batteries, other computing devices 595 (e.g., remote computing devices), a network service and the like. Examples of communication systems 580 include, but are not limited to, radio frequency (RF) transmitter, receiver, and/or transceiver circuitry, a Controller Area Network (CAN) bus, a universal serial bus (USB), parallel, and/or serial ports.

The computing device 500 may also have one or more input devices and/or one or more output devices shown as input/output devices 585. These input/output devices 585 may include a keyboard, a sound or voice input device, a touch, force and/or swipe input device, a display, speakers, etc. The aforementioned devices are examples and others may be used.

The computing device 500 may also include one or more sensors 590. The sensors 590 may be used to detect or otherwise determine current environmental condition information in a geographic area in which the computing device 500 is located.

The term computer-readable media as used herein may include computer storage media. Computer storage media may include volatile and nonvolatile, removable and non-removable media implemented in any method or technology for storage of information, such as computer readable instructions, data structures, or program modules.

The system memory 520, the removable storage 560, and the non-removable storage 570 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 500. Any such computer storage media may be part of the computing device 500. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method, comprising:
receiving, at a computing system, at least one health metric associated with a patient;
receiving, at the computing system, information from a patient computing device corresponding to a meal box fulfillment method;
categorizing the patient into a nutritional needs category based, at least in part, on the at least one health metric, the nutritional needs category being defined by nutritional criteria;
determining a list of meals meeting the nutritional criteria of the nutritional needs category;
providing the list of meals to the patient computing device;
receiving, at the computing system, information from the patient computing device corresponding to selected meals from the list of meals;
based, at least in part, on receiving information corresponding to the selected meals, providing the information corresponding to the selected meals to a meal box fulfillment center computing device; and
receiving, at the computing system, a meal box fulfillment confirmation message from the meal box fulfillment center computing device, the confirmation message including the information corresponding to the selected meals and an estimated shipment time of the meal box containing the selected meals.

2. The method of claim 1, wherein the nutritional needs category is a first nutritional needs category, the method further comprising:
receiving, at the computing system, at least one updated health metric after the patient has consumed at least one selected meal in the meal box; and
categorizing the patient into a second nutritional needs category based, at least in part, on the at least one updated health metric.

3. The method of claim 2, wherein nutritional criteria associated with the second nutritional needs category is different than the nutritional criteria associated first nutritional needs category.

4. The method of claim 1, wherein the at least one health metric is based, at least in part, on received prescription data associated with the patient.

5. The method of claim 1, wherein the at least one health metric comprises at least one disease state, age, gender, race, ethnicity, height, and weight.

6. The method of claim 5, wherein the disease state is one or more of cardiovascular disease, Coronary disease, atrial fibrillation, heart failure, dyslipidemia, cardio metabolic disease, metabolic disease, type I diabetes, type II diabetes, obesity, hypertension, rheumatoid arthritis, osteoarthritis, orthopedic diseases, endocrine diseases, renal diseases, cancer, gut dysfunction, immune diseases, auto-immune diseases, genetic diseases, inflammatory diseases, gastrointestinal diseases, neurological diseases, urological diseases, cancers, pulmonary diseases, and dermatological diseases.

7. The method of claim 1, wherein the list of meals is based, at least in part, on a combination of a determined geographic location of the patient and the information corresponding to the meal box fulfillment method.

8. The method of claim 1, wherein the information corresponding to the meal box fulfillment method comprises a selection of at least one of delivery to a delivery address or pick-up from an authorized pick-up location.

9. The method of claim 1, further comprising:
receiving, at the computing system, a consumption message indicating the patient has consumed at least one meal from the meal box, the consumption message comprising a date and a time of consumption for the at least one meal.

10. The method of claim 1, further comprising:
providing a report to an insurance coverage provider, wherein the report comprises a cost associated with the meal box.

11. A system, comprising:
a processor; and
a memory communicatively coupled to the processor and storing instructions that, when executed by the processor, perform operations, comprising:
receiving, at a computing system, at least one health metric associated with a patient;
receiving, at the computing system, information from a patient computing device corresponding to a meal box fulfillment method;
categorizing the patient into a nutritional needs category based, at least in part, on the at least one health metric, the nutritional needs category being defined by nutritional criteria;
determining a list of meals meeting the nutritional criteria of the first nutritional needs category;
providing the list of meals to the patient computing device;
receiving, at the computing system, information from the patient computing device corresponding to selected meals from the list of meals;
based, at least in part, on receiving information corresponding to the selected meals, providing the information corresponding to the selected meals to a meal box fulfillment center computing device; and
receiving, at the computing system, a meal box fulfillment confirmation message from the meal box fulfillment center computing device, the confirmation message including the information corresponding to the selected meals and an estimated shipment time of the meal box containing the selected meals.

12. The system of claim 11, wherein the memory communicatively coupled to the processor and storing instructions that, when executed by the processor, perform operations, further comprising:
receiving, at the computing system, at least one updated health metric after the patient has consumed at least one selected meal in the meal box; and
categorizing the patient into a second nutritional needs category based, at least in part, on the at least updated health metric.

13. The system of claim 12, wherein nutritional criteria associated with the second nutritional needs category is different than the nutritional criteria associated first nutritional needs category.

14. The system of claim 11, wherein the at least one health metric is based, at least in part, on received prescription data associated with the patient.

15. The system of system 11, wherein the at least one health metric comprises at least one disease state, age, gender, race, ethnicity, height, and weight.

16. The system of claim 15, wherein the disease state is one or more of cardiovascular disease, inclusive of peripheral arterial disease and electrophysiological diseases, Coronary disease, atrial fibrillation, heart failure, dyslipidemia, cardio metabolic disease, metabolic disease, type I diabetes, type II diabetes, obesity, hypertension, rheumatoid arthritis, osteoarthritis, orthopedic diseases, endocrine diseases, renal diseases, cancer, gut dysfunction, immune diseases, auto-immune diseases, genetic diseases, inflammatory diseases, gastrointestinal diseases, neurological diseases, urological diseases, cancers, pulmonary diseases, and dermatological diseases.

17. The system of claim 11, wherein the list of meals is based, at least in part, on a combination of a determined geographic location of the patient and the information corresponding to the meal box fulfillment method.

18. The system of claim 11, wherein the information corresponding to the meal box fulfillment method comprises a selection of at least one of delivery to a delivery address or pick-up from an authorized pick-up location.

19. The system of claim 11, wherein the memory communicatively coupled to the processor and storing instructions that, when executed by the processor, perform operations, further comprising:
receiving, at the computing system, a consumption message indicating the patient has consumed at least one meal from the meal box, the consumption message comprising a date and a time of consumption for the at least one meal.

20. A method, comprising:
receiving, at a computing system, at least one health metric associated with a patient;
receiving, at the computing system, information from a patient computing device corresponding to a meal box fulfillment method;
categorizing the patient into a first nutritional needs category based, at least in part, on the at least one health metric, the nutritional needs category being defined by nutritional criteria;
determining a list of meals meeting the nutritional criteria of the first nutritional needs category;
based, at least in part on the selected meal box fulfillment method, altering the list of meals;
providing the list of meals to a patient computing device;
based, at least in part, on receiving, at the computing system, information corresponding to selected meals from the list of meals, updating the list of meals provided to the patient computing device;
receiving, at the computing system, at least one updated health metric after the patient has consumed at least one selected meal in the meal box; and
categorizing the patient into a second nutritional needs category based, at least in part, on the at least one updated health metric.
